(19) United States
(12) Patent Application Publication
(10) Pub. No.: US 2018/0368896 A1
(43) Pub. Date: Dec. 27, 2018

(54) RIB FIXATION DEVICE

(71) Applicant: Ledford Lee Powell, Newport Beach, CA (US)

(72) Inventor: Ledford Lee Powell, Newport Beach, CA (US)

(21) Appl. No.: 15/692,057

(22) Filed: Aug. 31, 2017

Related U.S. Application Data
(60) Provisional application No. 62/523,670, filed on Jun. 22, 2017.

(51) Int. Cl. A61B 17/80

(52) U.S. Cl. CPC A61B 17/8076; A61B 17/8085

(57) ABSTRACT

A device for reducing and achieving fixation of a rib fracture may include a rib clip designed to partially encircle a rib bone without obstructing the intercostals nerve, wherein the rib clip is partially U-shaped. The rib clip may be built into a self-fixating plate. Alternatively, the rib clip may removably attach to an existing rib plate and, thus, be a standalone rib clip. The standalone rib clip may include a clip orifice extending therethrough to provide for attachment to the existing rib plate.

### RELATED APPLICATION

This application claims priority to provisional patent application U.S. Ser. No. 62/523,670 filed on Jun. 22, 2017, the entire contents of which is herein incorporated by reference.

### BACKGROUND

The embodiments herein relate generally to medical devices, and more particularly, to a rib plate that allows for self-reduction and fixation along with, in some cases, a separate be attachable rib clip that can be used in conjunction with an implantable rib plate.

Over 2.1 million people are admitted each year to hospitals after a fall or accident. Rib fractures are the most common injury after a fall. Rib fractures can cause pain, days off from work, long hospital stays, and even death. Early fixation of rib fractures improves patient outcomes, meaning that patients spend less time on the ventilator, less time in the ICU, less time in the hospital, have fewer pain medication requirements, and return to work and activities of daily living sooner. However, management of rib fractures has been challenging.

In particular, the existing devices for managing and treating rib fractures have a variety of problems. First, existing treatments for rib fractures has required the use of a lot of instrumentation to reduce the fracture and apply a fixation plate. This requires large incisions for reduction and fixation. The larger the incision, the more destroyed tissue, more pain for the patient, and longer recovery times after surgery or trauma. Additionally, some existing devices violate the intercostals nerve by placing a clamp below the rib and cause excessive disruption of the intercostals muscles because of wide fixation devices or multiple prongs above and below the rubs. Moreover, the existing devices can lead to increased discomfort to the patient, which may require hardware to be removed at a second operation.

Some conventional systems have poor outcomes, because they do not have reliable and consistent results in the hands of various surgeons. Additionally, some systems lead to poor fixation, because they do not use a plate and screw technology, which is most reliable. The existing systems that do not use the plate and screw technology are more challenging to implant in the patient, which makes it less desirable to use by the surgeon.

Therefore, what is needed is a device that allows surgeons to reduce and achieve fixation of rib fractures and flails chests with a smaller and less invasive surgery, which is also easy and reproducible.

### SUMMARY

Some embodiments of the present disclosure include a device for reducing and achieving fixation of a rib fracture. The device may include a rib clip designed to partially encircle a rib bone without obstructing the intercostals nerve, wherein the rib clip is partially U-shaped. The rib clip may be built into a self-fixating plate. Alternatively, the rib clip may removably attach to an existing rib plate and, thus, be a standalone rib clip. The standalone rib clip may include a clip orifice extending therethrough to provide for attachment to the existing rib plate.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present disclosure, shown in use.

FIG. 2 is a perspective view of one embodiment of the present disclosure.

FIG. 3 is a section view of one embodiment of the present disclosure, taken along line 3-3 in FIG. 1.

FIG. 4 is a perspective view of one embodiment of the present disclosure.

FIG. 5 is an exploded view of one embodiment of the present disclosure.

FIG. 6 is a perspective view of one embodiment of the present disclosure.

FIG. 7 is a section view of one embodiment of the present disclosure, taken along ling 7-7 in FIG. 6.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to reduce and achieve fixation of a bone fracture, such as a rib fracture, and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.
a. Rib Clip
b. Rib Plate
c. Screws

The various elements of the device of the present disclosure may be related in the following exemplary fashion. It is not intended to limit the scope or nature of the relationships between the various elements and the following examples are presented as illustrative examples only.

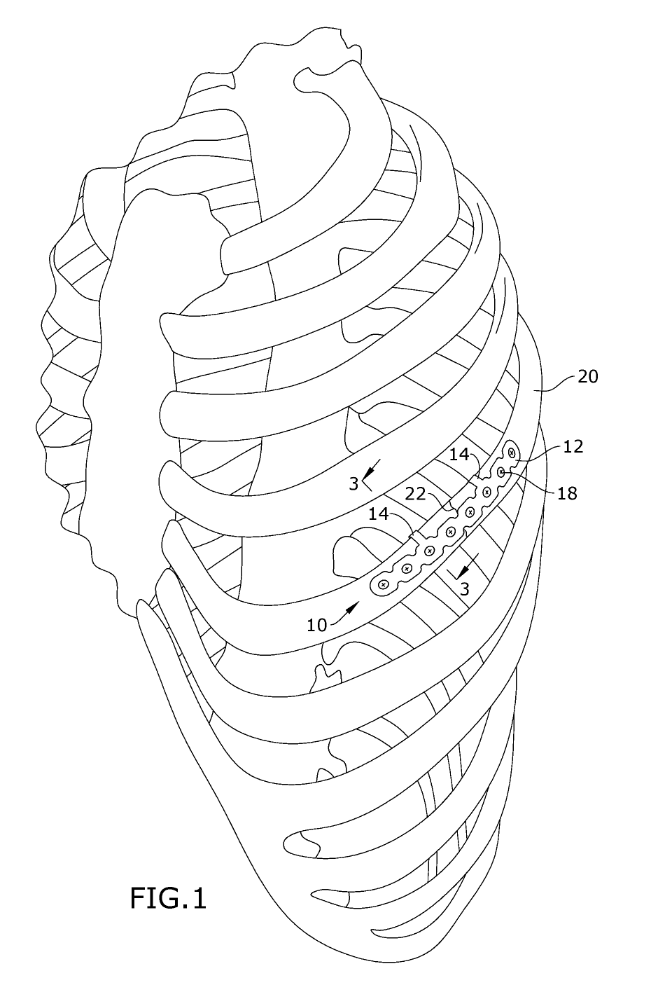

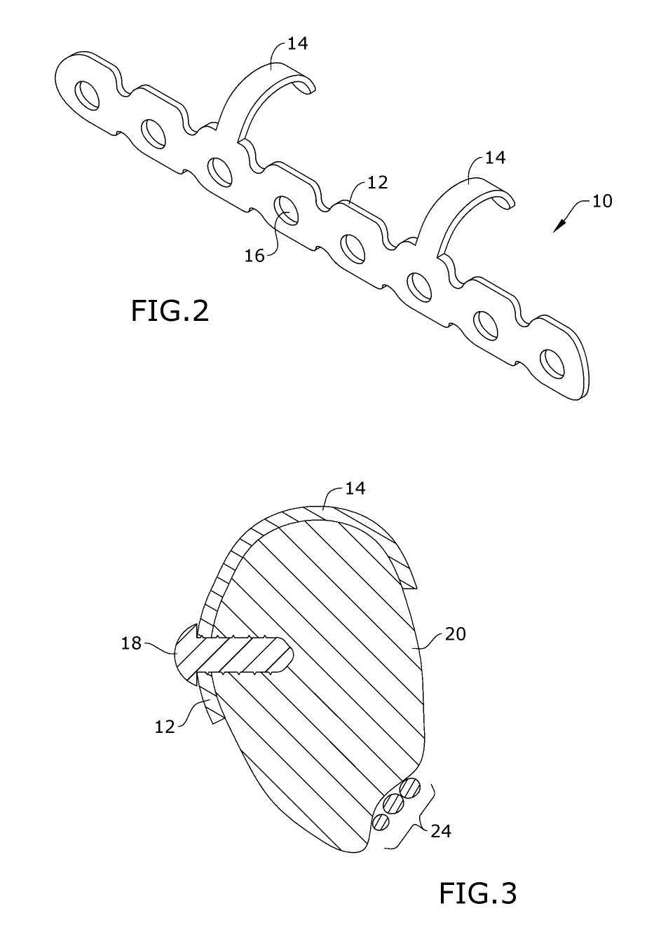

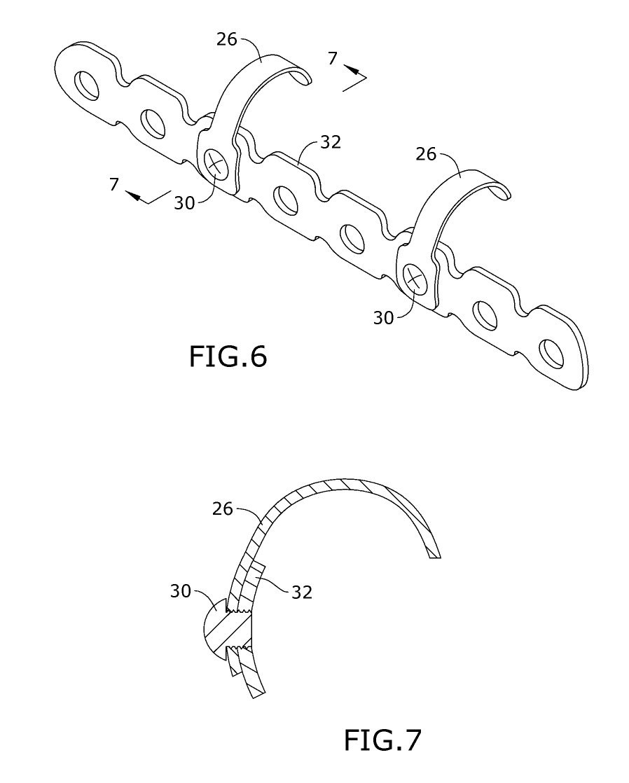

By way of example, and referring to FIGS. 1-7, some embodiments of the present disclosure include a device for reducing and achieving fixation of a rib fracture, the device comprising a rib clip designed to partially encircle a rib bone 20 without obstructing the intercostals nerve 24, wherein the rib clip may be partially U-shaped. The use of the rib clip may eliminate the need to use a reduction clamp during the surgical procedure.

In some embodiments, such as those shown in FIGS. 1-3, the rib clip 14 may be integrated into a self-fixating plate 10, such that the self-fixating plate 10 comprises an elongate perforated band 12 with a plurality of screw orifices 16, such as threaded screw orifices, extending therethrough, and at least one rib clip 14 extending outward from an edge of the perforated band 12, wherein the perforated band 12 is positioned adjacent and parallel to the rib bone 20 over a fracture line 22, a screw 18 is screwed into the rib bone 20 through each screw orifice 16, and the at least one rib clip 12 is positioned to partially encircle the rib bone 20 without obstructing the intercostals nerve 24. Thus, the device of the present disclosure may comprise a one-piece rigid construct of a perforated band 12 with at least one, such as two rib clips 12, incorporated into the band 12 as a single unit.

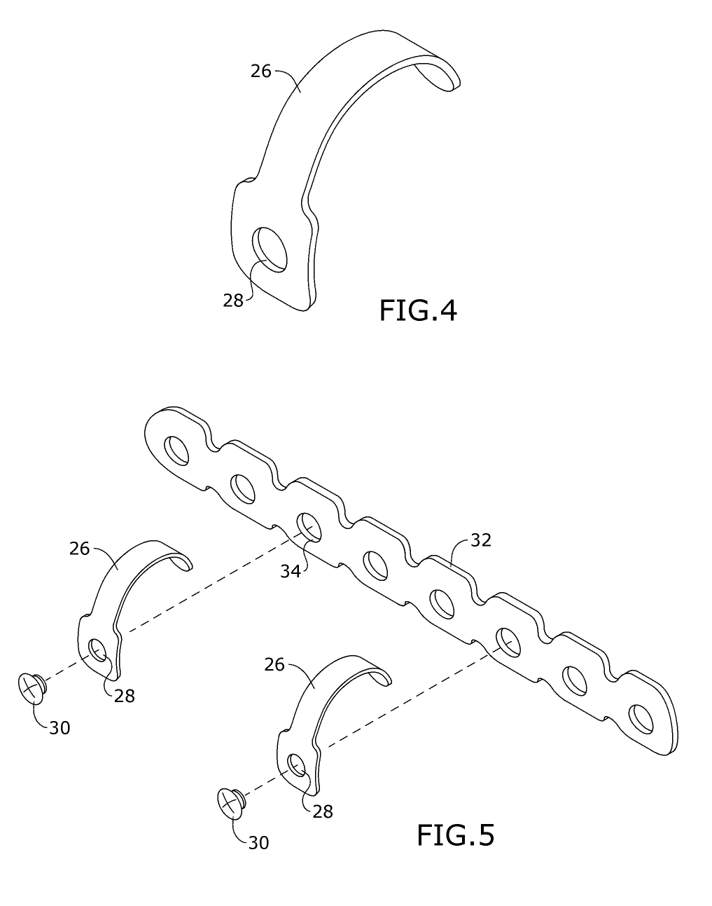

Alternatively, and as shown in FIGS. 4-7, some embodiments of the device include a standalone rib clip 26 designed to engage with an existing rib plate 32. For example, the standalone rib clip 26 may comprise a partially rounded U-shape with a clip screw orifice 28, such as a threaded clip screw orifice, at an end thereof, and the existing rib plate 32 may comprise a plurality of plate orifices 34, wherein the standalone rib clip 26 may be positioned such that the clip screw orifice 28 aligns with one of the plurality of plate orifices 34 and a clip screw 30 is inserted through the clip screw orifice 28 and the plate orifice 34, removably attaching the standalone rib clip 26 to existing rib plate 32. Thus, the standalone rib clip 26 may be attached to the existing rib plate 32 at any location along a length of the existing rib plate 32. The combination standalone rib clip 26 and attached existing rib plate 32 may then be positioned on a rib bone 20 such that the existing rib plate 32 crosses the fracture line 22 and the standalone rib clip 26 partially encircles the rib bone 20 without obstructing the intercostals nerve 24.

Additionally, the standalone rib clip 26 may be used in conjunction with the self-fixating plate 10, such that the rib bone 20 may be reduced and fixated with a perforated band 12 with built in rib clips 14 along with additional standalone rib clips 28 attached to the perforated band 12 via a clip screw 30.

The rib clip may provide for stabilization of the plate, while the plate screws 18 are used to fix the plate to the rib bone 20, resulting in a more reliable fixation with a less invasive approach using smaller incisions as compared to existing devices and treatment methods.

The device of the present disclosure may be made of any suitable material and, in some embodiments, comprises titanium metal. In a particular embodiment, the self-fixating plate 10 may be about 2 mm thick and about 8 cm long. The self-fixating plate 10 may have a gentle curve to mimic the contour of a rib bone 20, but may also be contoured using a device for bending the plate. The self-fixation plate 10 may have at least two rib clips 14 that are partially U-shaped extending from the top edge of the plate, wherein the rib clips 14 may be malleable and approximately 1 mm thick and 5 mm wide. The standalone rib clip 26 may be malleable and about 1 mm thick and 5 mm wide. The standalone rib clip 26 may widen at its base to approximately 1 cm wide and may have a clip orifice 28, such as a threaded clip orifice, extending therethrough, wherein the clip orifice 28 is used to screw the standalone rib clip 26 to a plate. The clip screw 30 may be about 3 mm long, such that it is long enough to attach the standalone rib clip 26 to the plate but not to the rib bone 20. Alternatively, a longer screw may be used to attach the system to the bone. Individual devices may be sterilized and packaged in a sterile fashion.

To use the device of the present disclosure, a surgeon may position the rib clips over the top of the rib bone with at least one clip on each side of the fracture line. This will hold the two ends of the broken bone in place. The rib clips also partially grasp the upper portion of the rib bone 20 to keep it stable for the next step. Thus, the rib clips take away the need for a reduction clamp, which is used in many operations to keep the bone edges from moving during fixation. Once the plate is applied to the broken rib bone, the plate is screwed into the broken rib bone using screws. If a rib clip is used separately form a rib plate, the surgeon may choose where the standalone rib clip will be placed on the rib plate. The rib clip is then screwed into the rib plate, and the rib plate with the newly attached rib clip is applied to the broken rib as described above.

While the device of the present disclosure is described as being used to reduce and achieve fixation of a rib bone, it may also be used to repair any broken bone in the body, wherein the shape and size of the rib clip and/or the plate may need to vary slightly to accommodate other bones.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A device for reducing and achieving fixation of a rib fracture, the device comprising:
a rib clip designed to partially encircle a rib bone without obstructing the intercostals nerve, wherein the rib clip is partially U-shaped.

2. The device of claim 1, further comprising a rib plate attached to the rib clip.

3. The device of claim 2, wherein:
the rib clip and the rib plate comprise a single construction with the rib clip extending from an outer edge of the rib plate; and
the rib plate comprises a plurality of screw orifices extending therethrough.

4. The device of claim 2, wherein:
the rib clip is a standalone rib clip having a clip orifice in an end thereof;
the rib plate comprises a plurality of screw orifices extending therethrough; and
a clip screw extends through the clip orifice and into one of the plurality of screw orifices, attaching the rib clip to the rib plate.

5. The device of claim 2, wherein the rib plate is elongate in shape and has a plurality of screw orifices extending therethrough.

6. The device of claim 1, wherein the rib clip comprises a titanium metal.

7. The device of claim 1, wherein the rib clip is about 1 mm thick and about 5 mm wide.
